(19) United States
(12) Patent Application Publication
(10) Pub. No.: US 2021/0395680 A1
(43) Pub. Date: Dec. 23, 2021

(54) COMPOSITION AND KIT FOR DIFFERENTIATION OF STEM CELLS INTO NEURAL PROGENITOR CELLS, AND METHOD USING SAME

(71) Applicant: CHA UNIVERSITY INDUSTRY-ACADEMIC COOPERATION FOUNDATION, Pocheon-si (KR)

(72) Inventor: Dong Youn HWANG, Seongnam-si (KR)

(73) Assignee: CHA UNIVERSITY INDUSTRY-ACADEMIC COOPERATION FOUNDATION, Pocheon-si (KR)

(21) Appl. No.: 17/290,085

(22) PCT Filed: Oct. 23, 2019
(86) PCT No.: PCT/KR2019/013931

§ 371 Date: Apr. 29, 2021

(30) Foreign Application Priority Data
Oct. 30, 2018 (KR) 10-2018-0131113

(51) Int. Cl. C12N 5/0793

(52) U.S. Cl. CPC C12N 5/0619; C12N 2501/727; C12N 2501/155; C12N 2506/03; C12N 2506/02; C12N 2506/45; C12N 2500/25

(57) ABSTRACT

Provided are a composition and a kit for differentiation of stem cells into neural progenitor cells (NPCs), each including a protein kinase C (PKC) inhibitor and a bone morphogenic protein (BMP) inhibitor, and a method using the same. Accordingly, stem cells may be efficiently differentiated into neural progenitor cells using a simple, low-cost composition.

### TECHNICAL FIELD

The present disclosure relates to a composition and a kit for differentiation of stem cells into neural progenitor cells (NPCs), each including a bone morphogenic protein (BMP) inhibitor, and a method using the same.

### BACKGROUND ART

Nervous system cells may be largely divided into two types, central nervous system (CNS) cells constituting the brain and spinal cord, and peripheral nervous system (PNS) cells constituting the motor, sensory, and autonomic neurons. Neurons, astrocytes, and oligodendrocytes that constitute the CNS (brain and spinal cord) may be produced by differentiating neural stem cells or neural progenitor cells (NPCs) which can be differentiated from pluripotent stem cells (PSCs), whereas the peripheral neurons (motor neurons, autonomic neurons, sensory neurons, etc.) and Schwann cells that constitute the PNS are derived from neural crest stem cells (NCSCs) which can be differentiated from PSCs. Therefore, CNS cells and PNS cells are produced from PSCs according to different differentiation pathways, through NPCs and NCSCs, respectively, and these different pathways are known to depend on the surrounding environment and intracellular signaling systems.

CNS diseases are largely divided into brain diseases and spinal cord diseases, and diseases caused by abnormal brain function include stroke, dementia, Parkinson's disease, etc. Spinal cord injury is caused by a sudden injury, and when the function of the spinal cord is impaired, problems such as loss of motor control, loss of sensation, and loss of bladder control may occur, and people with spinal cord injury live in pain throughout their lives. In addition, the number of people suffering from CNS diseases is rapidly increasing, as aging progresses around the world. Therefore, efforts to treat these diseases are urgently needed, because they deteriorate the quality of life of patients and their families and impose a great socio-economic burden on society.

Accordingly, to develop a cell replacement therapy that is considered the most fundamental treatment for CNS diseases, it is necessary to establish a method of efficiently differentiating PSCs into NPCs, which are parent cells of the CNS cells.

### DESCRIPTION OF EMBODIMENTS

### Technical Problem

There is provided a composition for differentiation of stem cells into neural progenitor cells.

There is provided a kit for differentiation of stem cells into neural progenitor cells.

There is provided a method of differentiating stem cells into neural progenitor cells.

### Solution to Problem

An aspect provides a composition for differentiation of stem cells into neural progenitor cells (NPCs), the composition including a protein kinase C (PKC) inhibitor and a bone morphogenic protein (BMP) inhibitor.

The term “PKC” refers to one of protein kinase enzymes that are involved in controlling functions of proteins through phosphorylation of hydroxyl groups of serine and threonine of proteins. PKC may be activated by signals such as increases in the concentration of diacylglycerol (DAG) or calcium ions (Ca²⁺). The mammalian PKC consists of 11 different kinds of molecular species cPKC (α, βI, βII, γ), nPKC (δ, ε, η, θ, μ), and aPKC (ξ, λ) which are classified into three groups. The PKC may be PKC-βI or βII.

The PKC inhibitor may inhibit expression or activity of PKC. The PKC inhibitor may be a PKC-β inhibitor. The PKC inhibitor may be selected from the group consisting of 2-[1-(3-dimethylaminopropyl)-5-methoxy indol-3-yl]-3-(1H-indol-3-yl) maleimide; 3-(1-(3-imidazol-1-yl propyl)-1H-indol-3-yl)-4-anilino-1H-pyrrole-2,5-dione); (3-(1H-indol-3-yl)-4-[2-(4-methylpiperazin-1-yl)quinazolin-4-yl]pyrrole-2,5-dione); 3-{1-[3-(amidinothio)propyl]-1H-indol-3-yl}-3-(1-methyl-1H-indol-3-yl)maleimide methane sulfonate; 13-hydroxyoctadecadienoic acid; bisindolylmaleimide; 2,6-diamino-N-([1-oxotridecyl)-2-piperidinyl]methyl)hexanamide; 4′-demethylamino-4′-hydroxystaurosporine; and 3-(13-methyl-5-oxo-6,7-dihydro-5H-indolo[2,3-a] pyrrolo [3,4-c] carbazol-12(13H)-yl) propanenitrile.

The term “BMP” refers to a growth factor that induces formation of bone and cartilage. The BMP may be selected from the group consisting of BMP1, BMP2, BMP3, BMP4, BMP5, BMP6, BMP7, BMP8a, BMP8b, BMP10, BMP11, and BMP15.

The BMP inhibitor may be a low-molecular-weight compound or polypeptide that inhibits cellular signaling of BMP. The BMP inhibitor may be a BMP receptor inhibitor, for example, a BMP receptor type 1 inhibitor or a BMP receptor type 2 inhibitor. The BMP inhibitor may be a low-molecular-weight inhibitor or a polypeptide inhibitor. The BMP inhibitor may be selected from the group consisting of dorsomorphin ((6-[4-[2-(1-piperidinyl)ethoxy]phenyl]-3-(4-pyridinyl)-pyrazolo[1,5-a]pyrimidine); dorsomorphin homolog 1 (DMH1, 4-[6-[4-(1-methylethoxy)phenyl]pyrazolo[1,5-a]pyrimidin-3-yl]-quinoline); K 02288 (3-[(6-amino-5-(3,4,5-trimethoxyphenyl)-3-pyridinyl]phenol); LDN 212854 (5-(6-(4-(1-piperazinyl)phenyl)pyrazolo[1,5-a]pyrimidin-3-yl)quinolone); and Noggin polypeptide. The dorsomorphin may also be called Compound C or BML-275.

The composition may further include a cell culture medium selected from the group consisting of a Dulbecco Modified Eagle's Medium (DMEM), a DMEM/F12, a F-10 nutrient medium (Nutrient M), a minimum essential medium (MEM), an RPMI medium 1640, an Opti-MEM I reduced serum medium, an Iscove's Modified Dulbecco's Medium (IMDM), an alpha-MEM, and a neurobasal medium. The composition may further include one selected from the group consisting of a Ham's F12 nutrient mixture, a B27 supplement, an F-10 nutrient mixture, an F-12 nutrient mixture, an N2 supplement, an HT supplement, a G-5 supplement, a lipid supplement, a serum replacement, and an insulin-transferrin-selenium (ITS) supplement.

A concentration of the PKC inhibitor in the composition may be about 0.01 μM to about 20 μM, about 0.1 μM to about 20 μM, about 0.25 μM to about 19 μM, about 0.5 μM to about 18 μM, about 0.75 μM to about 17 μM, about 1 μM to about 16 μM, about 1.25 μM to about 15 μM, about 1.5 μM to about 14 μM, about 1.75 μM to about 13 μM, about 2 μM to about 12 μM, about 2.25 μM to about 11 μM, about 2.5 μM to about 10 μM, about 2.75 μM to about 9 μM, about 3 μM to about 8 μM, about 3.25 μM to about 7 μM, about 3.5 μM to about 6 μM, about 3.75 μM to about 5 μM, or about 4 μM to about 5 μM.

A concentration of the BMP inhibitor in the composition may be about 0.01 μM to about 20 μM, about 0.02 μM to about 19 μM, about 0.04 μM to about 18 μM, about 0.06 μM to about 17 μM, about 0.08 μM to about 16 μM, about 0.1 μM to about 15 μM, about 0.12 μM to about 14 μM, about 0.14 μM to about 13 μM, about 0.16 μM to about 12 μM, about 0.18 μM to about 11 μM, about 0.2 μM to about 10 μM, about 0.21 μM to about 9 μM, about 0.22 μM to about 8 μM, about 0.23 μM to about 7 μM, about 0.24 μM to about 6 μM, or about 0.25 μM to about 5 μM.

The composition may include a cell culture medium, an ITS supplement, the PKC inhibitor, and 0.25 μM to 5 μM of the BMP inhibitor. The composition may include a cell culture medium, a serum replacement, the PKC inhibitor, and 5 μM to 10 μM of the BMP inhibitor.

The concentrations of the components in the composition may be the final concentrations. The composition may be concentrated, dried, or diluted. For example, when the composition is concentrated 50-fold, the composition may be added to a medium including stem cells at a final concentration diluted 1/50.

The composition may be a composition for cell culture.

The term “stem cells” refers to totipotent cells that are able to differentiate into all types of cells or pluripotent cells that are able to differentiate into many types of cells, or multipotent cells that are able to differentiate into various kinds of cells, and stem cells which are undifferentiated cells may differentiate into cells of a specific tissue. The stem cells may be embryonic stem cells (ESCs), adult stem cells, induced pluripotent stem cells (iPSCs), or somatic cell nuclear transfer embryonic stem cells.

The embryonic stem cells refer to an in vitro culture of an extract of an inner cell mass of a blastocyst, which is formed immediately before implantation of a fertilized egg in the mother's uterus. The adult stem cells, which are undifferentiated cells that exist only in a small amount in each tissue of the body, refer to cells that replace dead or damaged tissue. The induced pluripotent stem cells (iPSCs) refer to cells that are induced to have pluripotency like embryonic stem cells by injecting cell dedifferentiation-related genes into somatic cells that have undergone differentiation and returning the cells to an early stage of totipotent stem cells. The induced pluripotent stem cells may be, for example, human dermal fibroblast-derived induced pluripotent stem cells (human dermal fibroblast-iPSCs: hDF-iPSCs), blood cell-derived induced pluripotent stem cells (blood cell-iPSCs), or urine cell-derived induced pluripotent stem (urine-iPSCs). The somatic cell nuclear transfer embryonic stem cells refer to totipotent cells obtained from an in vitro culture of an extract of an inner cell mass of a blastocyst, which is formed during the initial development of cells prepared by removing a nucleus of an egg and replacing the nucleus with a nucleus of a somatic cell.

The stem cells may be cells derived from a mammal, for example, a human, mouse, rat, ape, cow, horse, pig, dog, sheep, goat, or cat.

The term “NPCs” refers to cells capable of self-renewal and having ability to differentiate into a neural cell lineage. The NPCs may also be called neural stem cells (NSCs). The NPCs may differentiate into neurons, astrocytes, or oligodendrocytes.

The NPCs may be cells expressing SOX1. The NPCs may not express P75 protein. The P75 protein may be expressed in neural crest stem cells (NCSCs).

The term “differentiation” refers to a phenomenon in which a cell's structure or function is specialized during growth by cell division and proliferation. A totipotent, multipotent, or pluripotent stem cell may completely differentiate into a specific cell via a specific type of progenitor cell. The embryonic stem cells, adult stem cells, induced pluripotent stem cells, or somatic cell nuclear transfer embryonic stem cells may differentiate into the neural progenitor cells. The NPCs may differentiate into neurons, astrocytes, oligodendrocytes, etc.

The NPCs may be used in the treatment of CNS diseases. The CNS diseases are, for example, stroke, dementia, Parkinson's disease, Huntington's disease, and multiple sclerosis. The CNS diseases may be spinal cord injury caused by injury.

The composition may be a single composition or a separate composition.

Another aspect provides a kit for differentiation of stem cells into NPCs, the kit including the composition according to one aspect and a cell culture dish.

The composition, the stem cells, the NPCs, and the differentiation are the same as described above.

The cell culture dish refers to a cell culture vessel, and includes any cell culture vessel regardless of the material, size, and shape of the culture dish.

The cell culture dish may be a culture dish for suspension culture or a culture dish for adherent culture. The culture dish for adherent culture may be coated with a polypeptide. The polypeptide may be a polypeptide for adhering or culturing stem cells. The polypeptide is, for example, vitronectin (VTN), laminine, fibronectin, poly ornithine, or Matrigel™.

Still another aspect provides a method of differentiating stem cells into NPCs, the method including suspension-culturing the stem cells in a cell culture medium containing a PKC inhibitor and a BMP inhibitor to obtain embryoid bodies (EBs); and adherent-culturing cells obtained by chopping the EBs to induce differentiation into NPCs.

The stem cell, the PKC, the PKC inhibitor, the BMP, the BMP inhibitor, the cell culture medium, the NPCs, and the differentiation are the same as described above.

The method may further include inoculating the stem cells in the cell culture dish. The stem cells may be inoculated in the presence of a basic culture medium.

The method includes suspension-culturing the stem cells in a medium containing a BMP inhibitor to obtain EBs.

The suspension-culturing may be culturing without adhering the stem cells onto the bottom of the culture dish. When the stem cells are suspension-cultured, EBs may be formed, which are a cell mass formed by aggregation of stem cells into a ball shape at the beginning of cell division.

In the method, the culture time of the stem cells may vary depending on the culture conditions. The stem cells may be cultured for, for example, about 1 day to about 10 days, about 1 day to about 9 days, about 1 day to about 8 days, about 1 day to about 7 days, about 1 day to about 6 days, about 1 day to about 5 days, about 1 day to about 4 days, about 2 days to about 4 days, about 3 days to about 4 days, or about 4 days. In the method, the stem cells may be cultured at about 30° C. to about 40° C., about 30° C. to about 37° C., or about 37° C.

The method includes adherent-culturing (attachment culturing) cells obtained by chopping the EBs to induce differentiation into NPCs.

The adherent-culturing may be culturing by adhering cells onto the bottom of the culture dish.

The adherent-culturing may be performed for about 1 day to about 15 days, about 1 day to about 14 days, about 1 day to about 13 days, about 1 day to about 12 days, about 1 day to about 11 days, about 1 day to about 10 days, about 1 day to about 9 days, about 1 day to about 8 days, about 1 day to about 7 days, about 1 day to about 6 days, about 1 day to about 5 days, or about 2 days to about 5 days.

The medium may further include a PKC inhibitor.

A proportion of NPCs in the cells cultured by the method may be about 50% or more, about 60% or more, about 70% or more, about 80% or more, about 85% or more, about 90% or more, about 95% or more, about 97% or more, about 98% or more, or about 99% or more.

### Advantageous Effects of Disclosure

According to a composition and a kit for differentiation of stem cells into neural progenitor cells (NPCs), each including a protein kinase C (PKC) inhibitor and a bone morphogenic protein (BMP) inhibitor, and a method using the same, stem cells may be efficiently differentiated into NPCs using a simple, low-cost composition.

### BRIEF DESCRIPTION OF DRAWINGS

FIG. 1A shows an illustration of a culture process of inducing differentiation of human PSCs into NPCs according to an aspect, FIG. 1B shows images of embryoid bodies (EBs) obtained by suspension culture for about 4 days in a defined medium 1 including a PKC-beta inhibitor (PKCI) and DMH1, and cells (rossette formed) obtained by adherent culture of the embryoid bodies for about 5 days (left: embryoid bodies, right: cells forming rosettes), and FIG. 1C shows plots showing results of flow cytometry for detecting SOX1 (NPC marker) and P75 (NCSC marker) in cells obtained by culturing for a total of 9 days;

FIGS. 2A, 2B, and 2C show images (top) of cells cultured for a total of 9 days, and plots showing results of flow cytometry for detecting SOX1 (middle) and P75 (bottom), when embryonic stem cell lines of CHA15-hESC and CHA6-hESC and an induced pluripotent stem cell line (iPS cell #3) were used, respectively;

FIG. 3A shows images of cells obtained by suspension culture for about 4 days and adherent culture for about 5 days in a defined medium 2 including a PKC-beta inhibitor (PKCI) and DMH1, and FIG. 3B shows plots showing results of flow cytometry for detecting SOX1 and P75 (left: flow cytometry plot of SOX1, right: flow cytometry plot of P75);

FIG. 4A shows images of cells obtained by suspension culture for about 4 days and adherent culture for about 5 days in a defined medium 3 including a PKC-beta inhibitor (PKCI) and DMH1, and FIG. 4B shows plots showing results of flow cytometry for detecting SOX1 and P75 (left: flow cytometry plot of SOX1, right: flow cytometry plot of P75);

FIG. 5A shows images of cells obtained by suspension culture for about 4 days and adherent culture for about 5 days in defined media 1 including a PKC-beta inhibitor (5 μM) and different concentrations of DMH1, and FIGS. 5B, 5C, and 5D show plots showing results of flow cytometry for detecting SOX1 and P75 (upper: flow cytometry plot of SOX1, lower: flow cytometry plot of P75); and

FIG. 6A shows images of cells obtained by suspension culture for about 4 days and adherent culture for about 5 days in defined media 2 including a PKC-beta inhibitor (5 μM) and different concentrations of DMH1, and FIGS. 6B, 6C, and 6D show plots showing results of flow cytometry for detecting SOX1 and P75 (upper: flow cytometry plot of SOX1, lower: flow cytometry plot of P75).

### MODE OF DISCLOSURE

Hereinafter, the present disclosure will be described in more detail with reference to exemplary embodiments. However, these exemplary embodiments are only for illustrating the present disclosure, and the scope of the present disclosure is not limited to these exemplary embodiments.

### Example 1. Differentiation of Human Pluripotent Stem Cells into Neural Progenitor Cells

1. Induction of Differentiation into Neural Progenitor Cells in Presence of PKC Inhibitor and BMP Inhibitor

(1) Use of Defined Medium 1 Including PKC Inhibitor and BMP Inhibitor

Colonies obtained by culturing H9 human embryonic stem cells (hESCs) (WiCell Research Institute, Inc. Madison, Wis., U.S.A.) in a culture medium were dissociated into single cells using accutase (Life Technologies).

The cells dissociated into single cells were seeded in a culture plate coated with a protein such as vitronectin (VTN) (vitronectin XF, STEMCELL Technologies), laminine (rhLaminin-521, Thermo Fisher Scientific Inc., Waltham, Mass., USA), fibronectin (Thermo Fisher Scientific Inc.), or Matrigel™ (StemCell Technologies, Inc.). An embryonic stem cell (ESC) culture medium was added to the seeded cells to perform undifferentiation culture. As the ESC culture medium, Essential 8 (E8) medium (StemCell Technologies Inc.), TeSR2 medium (StemCell Technologies Inc), StemMACS (Miltenyi Biotec, Bergisch Gladbach, Germany), etc. was used.

The obtained colonies were finely chopped and suspension-cultured under conditions of 37° C. and 5% CO₂ in a defined medium 1 containing 5 μg/ml of a PKC inhibitor and 0.5 μM of a BMP inhibitor for about 4 days to prepare embryoid bodies (EBs). The defined medium 1 included DMEM/F12 (Life Technologies), 10 μg/ml of insulin (Sigma-Aldrich), 9 μg/ml of transferrin (Sigma-Aldrich), and 14 ng/ml of sodium selenite (Sigma-Aldrich). 3-(1-(3-Imidazol-1-yl propyl)-1H-indol-3-yl)-4-anilino-1H-pyrrole-2,5-dione) (PKC-beta inhibitor) (Millipore) was used as the PKC inhibitor. DMH1 (dorsomorphin homolog 1) (Tocris Bioscience, USA) which is a compound inhibiting BMP 1 type receptor was used as the BMP inhibitor. As a negative control, dimethyl sulfoxide (DMSO) (Sigma-Aldrich) was used instead of the drug.

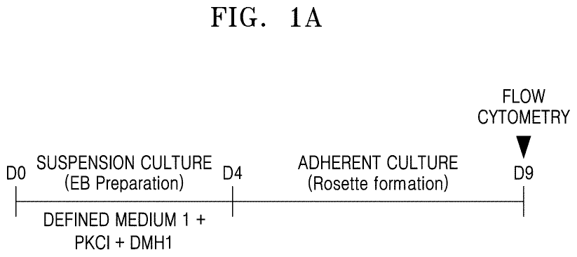

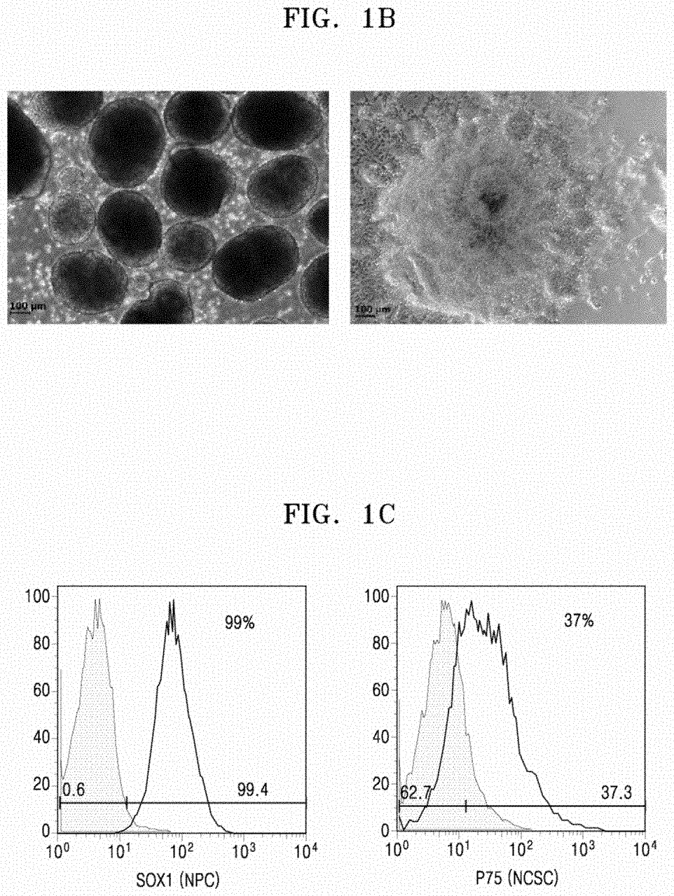

EBs, which are round cell masses produced by suspension culture (defined medium 1) for about 4 days, were finely chopped, and then adhered to the bottom of the culture plate, followed by adherent culture for about 5 days (DMEM-F12, 1×N2 supplement, 25 μg/ml of insulin, 20 ng/ml of bFGF, and a matrigel-coated culture plate were used). Microscopic images of EBs obtained by suspension culture for about 4 days, and cells forming rossettes obtained by adherent culture for about 5 days are shown in FIG. 1B (left: embryoid bodies, right: cells forming rosettes).

2% (v/v) paraformaldehyde (Sigma-Aldrich) was added to cells cultured for a total of 9 days (suspension culture for 4 days+adherent culture for 5 days), and allowed to react at room temperature for about 10 minutes to fix the cells. 2% (v/v) normal serum/1×PBS (Vector Laboratories, Inc., Burlingame, Calif.) containing 0.1% (v/v) TRITON™ X-100 (Sigma-Aldrich) was added to the cells, and allowed to react at room temperature for 30 minutes to block the fixation. To analyze a proportion of cells expressing SOX1 which is a neural progenitor cell (NPC) marker or P75 protein which is a neural crest stem cell (NCSC) marker, immunostaining was performed using phycoerythrin (PE)-labeled anti-P75 monoclonal antibody (1:50 dilution) (Miltenyi Biotec), and phycoerythrin (PE)-labeled anti-Sox1 monoclonal antibody (1:100 dilution) (BD Biosciences). The immunostained cells were subjected to flow cytometry using a BD FACSCalibur flow cytometer (BD Biosciences, Sparks, Md., USA). The results of flow cytometry are shown in FIG. 1C.

As shown in FIG. 1C, it was confirmed that when hESCs were cultured in the defined medium 1 including the PKC inhibitor and DMH1 by the method of differentiating via embryoid bodies, most of the cultured cells expressed SOX1 which is a NPC marker, and the number of cells expressing P75 which is a neural crest stem cell marker was small. Therefore, it was confirmed that when hESCs are cultured in the defined medium 1 including the PKC inhibitor and DMH1, differentiation into NPC is induced.

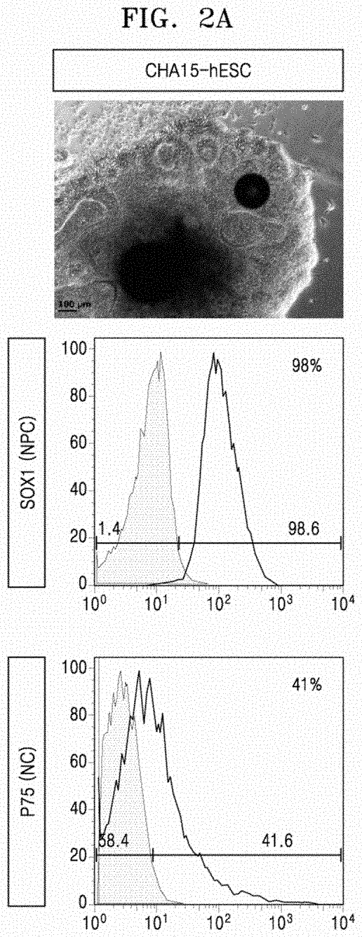

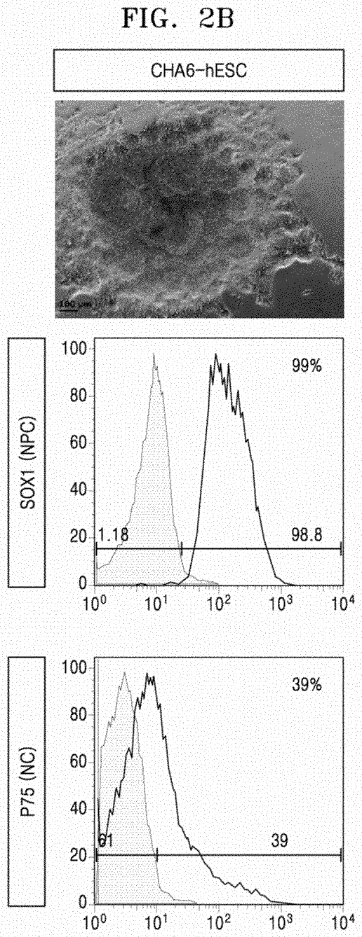

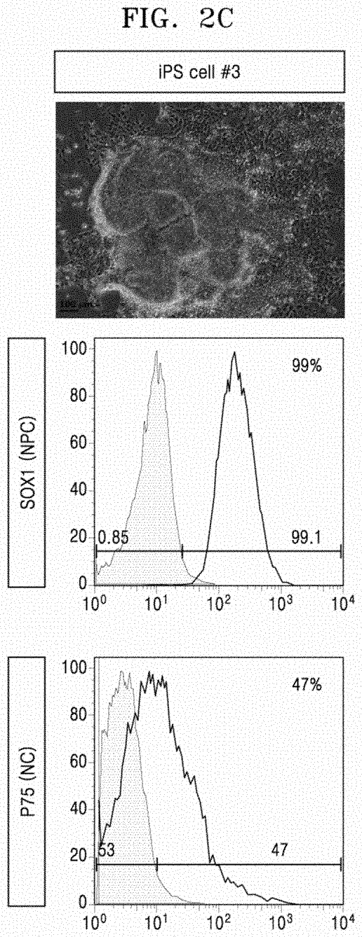

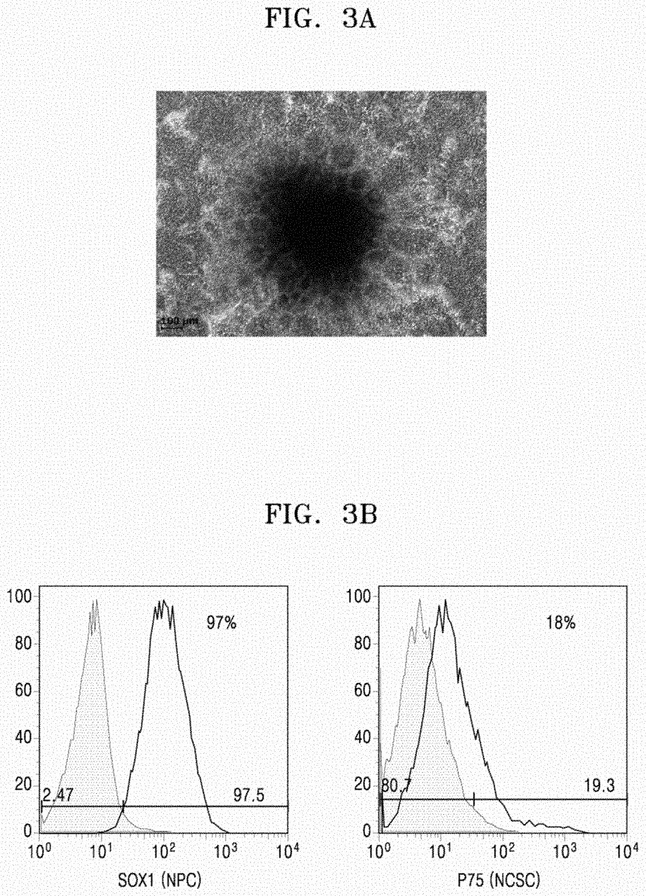

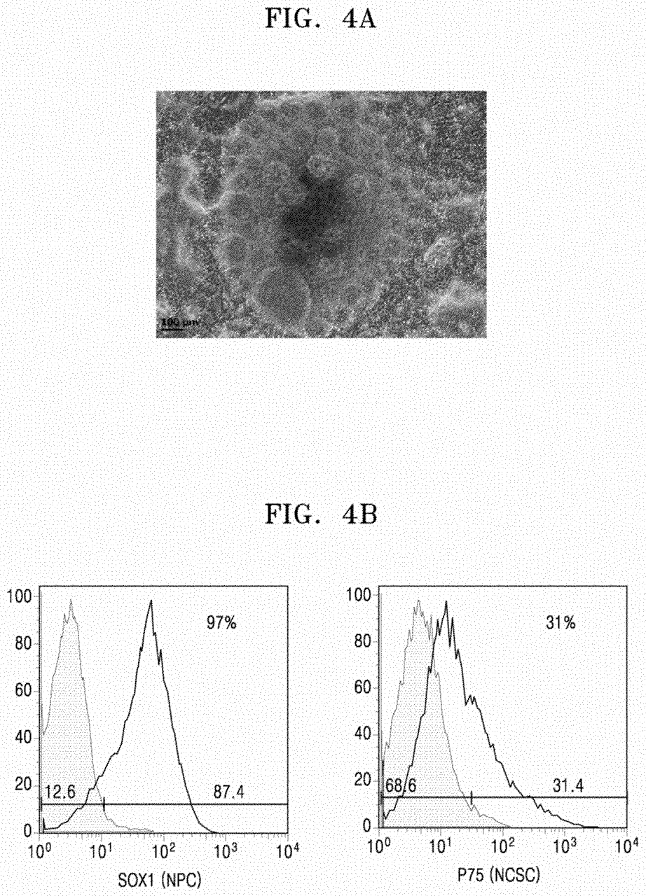

Further, two different kinds of embryonic stem cell lines (CHA15-hESC and CHA6-hESC) (CHA stem cell Institute) and one kind of induced pluripotent stem cell line (iPS cell #3) (prepared by a known method) were used, instead of H9 hESCs, and images (upper) of the cells cultured for a total of 9 days, results of SOX1 flow cytometry (middle), and results of P75 flow cytometry (lower) are shown in FIGS. 2A, 2B, and 2C. As shown in FIGS. 2A, 2B, and 2C, it was confirmed that when stem cells are cultured in the defined medium 1 including the PKC inhibitor and the BMP inhibitor, differentiation into NPCs is induced.

(2) Use of Defined Medium 2 Including PKC Inhibitor and BMP Inhibitor

H9-hESCs were cultured by the method of differentiating via embryoid bodies as in Example 1.1(1). However, a defined medium 2 described below was used instead of the defined medium 1 used in Example 1.1(1).

The defined medium 2 was prepared by mixing knockout DMEM (Invitrogen), 20% (v/v) knockout serum replacement (KSR, Invitrogen), 2 mM L-glutamine (invitrogen), 1% (w/v) penicillin/streptomycin (Invitrogen), and 0.1 mM β-mercaptoethanol (Invitrogen).

Cells obtained by suspension culture for about 4 days and adherent culture for about 5 days (DMEM-F12, 1×N2 supplement, 25 μg/ml of insulin, 20 ng/ml of bFGF, and a matrigel-coated culture plate were used) were examined using a microscope, and images thereof are shown FIG. 3A.

In the obtained cells, the number of SOX1-expressing cells (i.e., NPCs) and the number of P75-expressing cells (i.e., neural crest stem cells) were determined by flow cytometry, and the results are shown in FIG. 3B.

As shown in FIG. 3B, it was confirmed that when hESCs were cultured in the defined medium 2 including the PKC inhibitor, most of the cultured cells expressed SOX1 which is a NPC marker, and the number of cells expressing P75 which is a neural crest stem cell marker was relatively very small. Therefore, it was confirmed that when hESCs are cultured in the defined medium 2 including the PKC inhibitor and BMP inhibitor, differentiation into NPCs is induced.

(3) Use of Defined Medium 3 Including PKC Inhibitor and BMP Inhibitor

H9-hESCs were cultured by the method of differentiating via embryoid bodies as in Example 1(1). However, a defined medium 3 was used instead of the defined medium 1 used in Example 1(1). The defined medium 3 was prepared by mixing DMEM/F12 (Invitrogen) and N2 supplement (Invitrogen).

Cells obtained by suspension culture for about 4 days and adherent culture for about 5 days (DMEM-F12, 1×N2 supplement, 25 μg/ml of insulin, 20 ng/ml of bFGF, and a matrigel-coated culture plate were used) were examined using a microscope, and images thereof are shown FIG. 4A.

In the obtained cells, the number of SOX1-expressing cells (i.e., NPCs) and the number of P75-expressing cells (i.e., neural crest stem cells) were determined by flow cytometry, and the results are shown in FIG. 4B.

As shown in FIG. 4B, it was confirmed that when hESCs were cultured in the defined medium 3 including the PKC inhibitor, most of the cultured cells expressed SOX1 which is a NPC marker, and the number of cells expressing P75 which is a neural crest stem cell marker was relatively very small. Therefore, it was confirmed that when hESCs are cultured in the defined medium 3 including the PKC inhibitor and BMP inhibitor, differentiation into NPCs is induced.

2. Induction of Differentiation into Neural Progenitor Cells in Presence of PKC Inhibitor and BMP Inhibitor

(1) Use of Defined Medium 1 Including PKC Inhibitor and BMP Inhibitor

H9-hESCs were cultured by the method of differentiating via embryoid bodies as in Example 1.1(1). However, 5 μM of the PKC inhibitor and 0 μM to 5 μM of the BMP inhibitor (DMH1) were added to the defined medium 1 in Example 1.1(1).

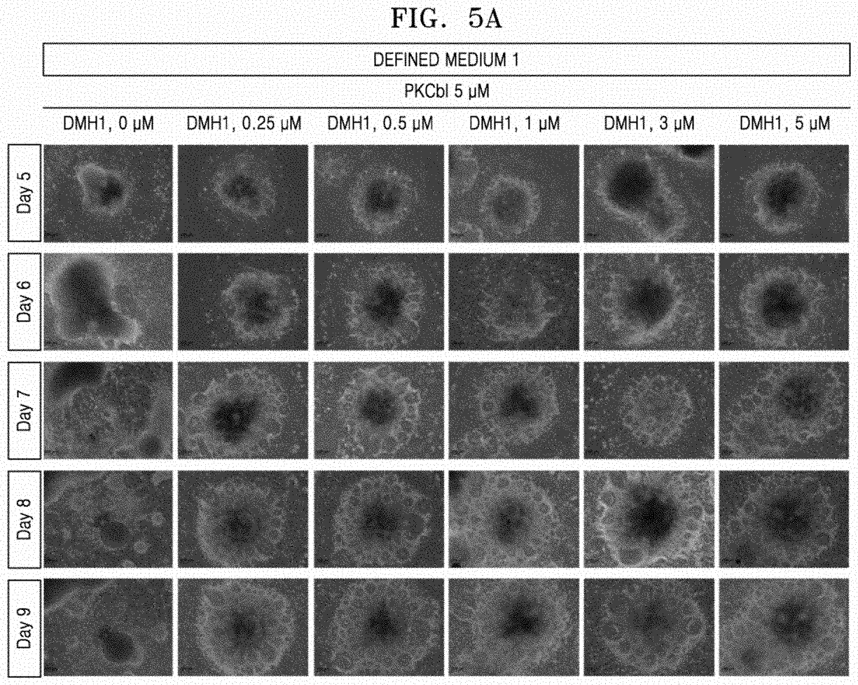

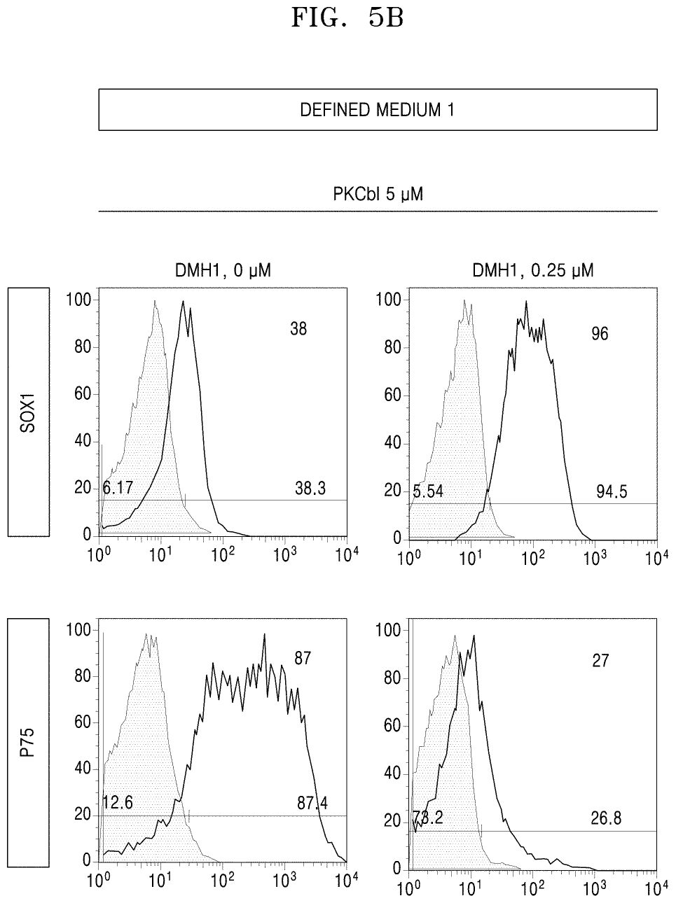

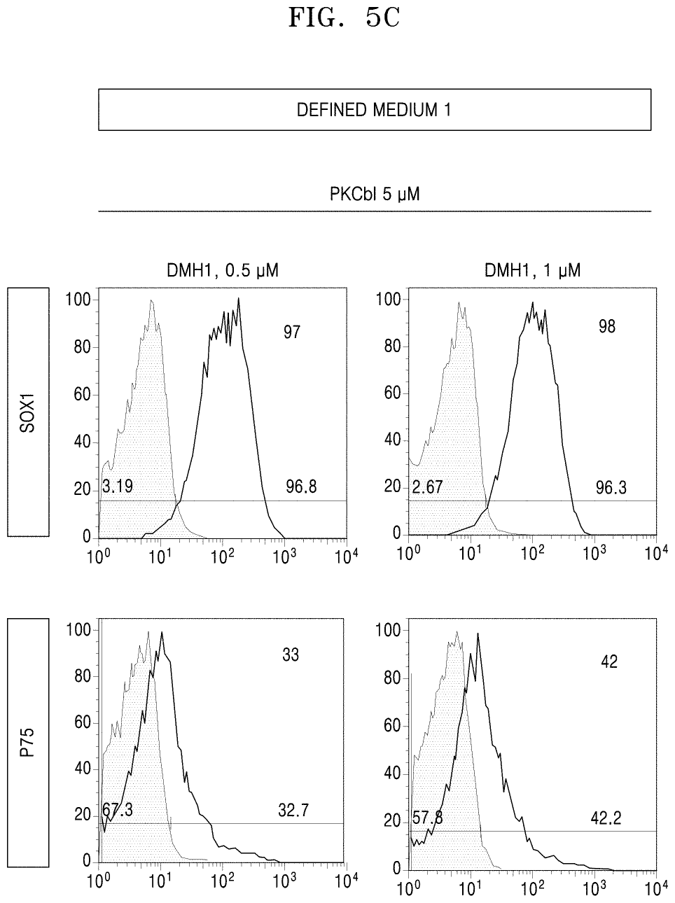

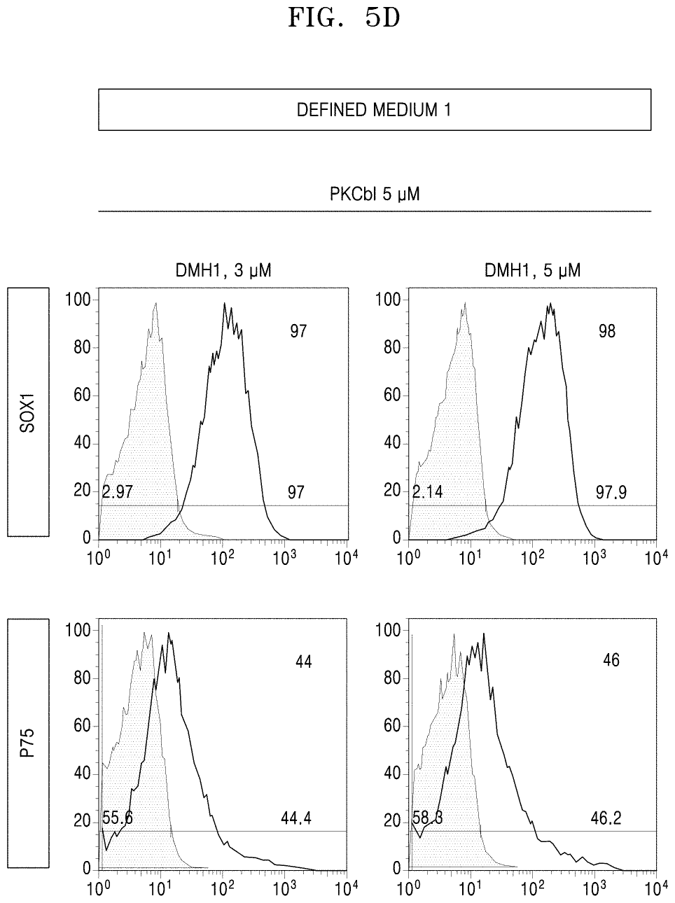

Cells obtained by suspension culture for about 4 days and adherent culture for about 5 days were examined using a microscope, and images thereof are shown FIG. 5A. In the obtained cells, the number of SOX1-expressing cells (i.e., NPCs) and the number of P75-expressing cells (i.e., neural crest stem cells) were determined by flow cytometry, and the results are shown in FIGS. 5B, 5C, and 5D.

As shown in FIGS. 5A to 5D, it was confirmed that when H9-hESCs were cultured in the defined medium 1 including 5 μM of the PKC inhibitor and 0.25 μM to 5 μM of DMH1, differentiation into SOX1-expressing NPCs (about 96% or more) was observed. In contrast, it was confirmed that when H9-hESCs were cultured in the defined medium 1 including only 5 μM of the PKC inhibitor without DMH1, differentiation into P75-expressing neural crest stem cells (about 87% or more) was observed.

(2) Use of Defined Medium 2 Including PKC Inhibitor and BMP Inhibitor

H9-hESCs were cultured as described in Example 1.2(1). However, the defined medium 2 was used instead of the defined medium 1.

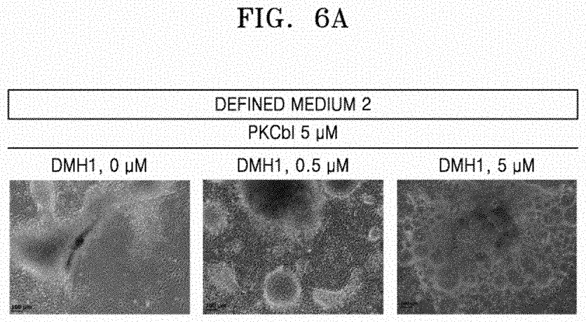

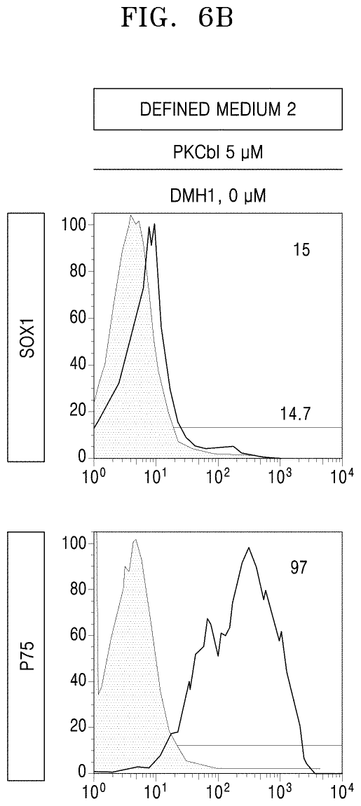

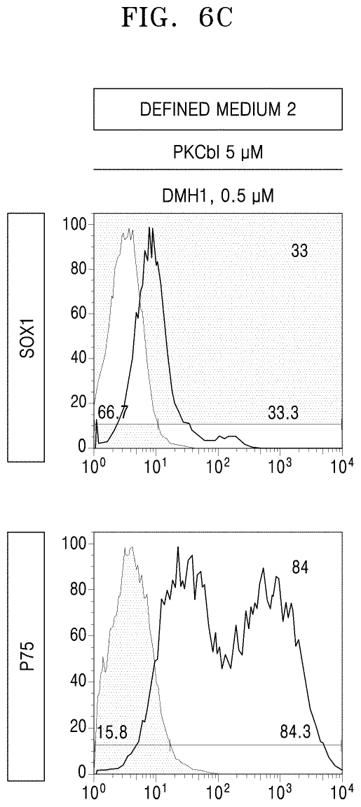

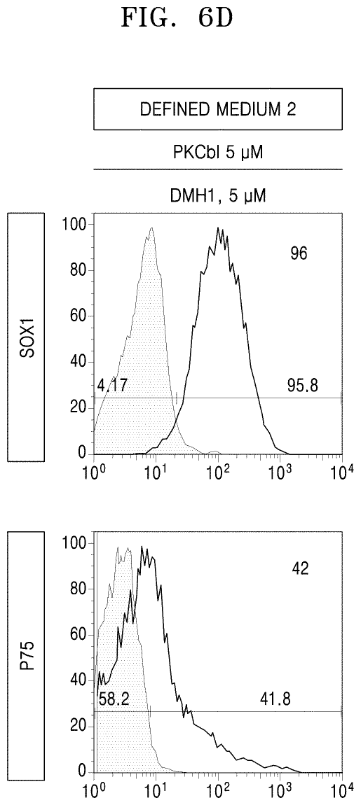

Cells obtained by suspension culture for about 4 days and adherent culture for about 5 days were examined using a microscope, and images thereof are shown FIG. 6A. In the obtained cells, the number of SOX1-expressing cells (i.e., NPCs) and the number of P75-expressing cells (i.e., neural crest stem cells) were determined by flow cytometry, and the results are shown in FIGS. 6B, 6C, and 6D.

As shown in FIGS. 6A to 6D, it was confirmed that when H9-hESCs were cultured in the defined medium 2 including 5 μM of the PKC inhibitor and 5 μM of DMH1, differentiation into SOX1-expressing NPCs (about 96% or more) was observed. In contrast, it was confirmed that when H9-hESCs were cultured in the defined medium 2 including 5 μM of the PKC inhibitor and 0 μM to 0.5 μM of DMH1, differentiation into P75-expressing neural crest stem cells (about 84% or more) was observed.

1. A composition for differentiation of stem cells into neural progenitor cells (NPCs), the composition comprising a protein kinase C (PKC) inhibitor and a bone morphogenic protein (BMP) inhibitor.

2. The composition of claim 1, wherein the PKC inhibitor is a PKC-3 inhibitor.

3. The composition of claim 1, wherein the PKC-3 inhibitor is selected from the group consisting of:
2-[1-(3-dimethylaminopropyl)-5-methoxy indol-3-yl]-3-(1H-indol-3-yl) maleimide;
3-(1-(3-imidazol-1-yl propyl)-1H-indol-3-yl)-4-anilino-1H-pyrrole-2,5-dione;
(3-(1H-indol-3-yl)-4-[2-(4-methylpiperazin-1-yl)quinazolin-4-yl]pyrrole-2,5-dione;
(3-{1-[3-(amidinothio)propyl]-1H-indol-3-yl}-3-(1-methyl-1H-indol-3-yl)maleimide methane sulfonate;
13-hydroxyoctadecadienoic acid;
bisindolylmaleimide;
2,6-diamino-N-([1-oxotridecyl)-2-piperidinyl]methyl)hexanamide;
4′-demethylamino-4′-hydroxystaurosporine; and
3-(13-methyl-5-oxo-6,7-dihydro-5H-indolo[2,3-a] pyrrolo [3,4-c] carbazol-12(13H)-yl) propanenitrile.

4. The composition of claim 1, wherein the BMP inhibitor is selected from the group consisting of:
dorsomorphin (6-[4-[2-(1-piperidinyl)ethoxy]phenyl]-3-(4-pyridinyl)-pyrazolo[1,5-a]pyrimidine);
dorsomorphin homolog 1 (DMH1, 4-[6-[4-(1-methylethoxy)phenyl]pyrazolo[1,5-a]pyrimidin-3-yl]-quinoline);
K 02288 (3-[(6-amino-5-(3,4,5-trimethoxyphenyl)-3-pyridinyl]phenol);
LDN 212854 (5-(6-(4-(1-piperazinyl)phenyl)pyrazolo[1,5-a]pyrimidin-3-yl)quinolone); and
Noggin polypeptide.

5. The composition of claim 1, further comprising a cell culture medium selected from the group consisting of a Dulbecco Modified Eagle's Medium (DMEM), a DMEM/F12, a F-10 nutrient medium (Nutrient M), a minimum essential medium (MEM), an RPMI medium 1640, an Opti-MEM I reduced serum medium, an Iscove's Modified Dulbecco's Medium (IMDM), an alpha-MEM, and a neurobasal medium.

6. The composition of claim 5, further comprising one selected from the group consisting of a Ham's F12 nutrient mixture, a B27 supplement, an F-10 nutrient mixture, an F-12 nutrient mixture, an N2 supplement, an HT supplement, a G-5 supplement, a lipid supplement, a serum replacement, and an insulin-transferrin-selenium (ITS) supplement.

7. The composition of claim 1, wherein a concentration of the PKC inhibitor in the composition is 0.1 μM to 20 μM.

8. The composition of claim 1, wherein a concentration of the BMP inhibitor in the composition is 0.01 μM to 20 μM.

9. The composition of claim 1, comprising a cell culture medium, an insulin-transferrin-selenium (ITS) supplement, the PKC inhibitor, and 0.25 μM to 5 μM of the BMP inhibitor.

10. The composition of claim 1, comprising a cell culture medium, a serum replacement, the PKC inhibitor, and 5 μM to 10 μM of the BMP inhibitor.

11. The composition of claim 1, wherein the stem cells are embryonic stem cells (ESCs), adult stem cells, induced pluripotent stem cells (iPSCs), or somatic cell nuclear transfer embryonic stem cells.

12. A kit for differentiation of stem cells into neural progenitor cells, the kit comprising the composition of claim 1 and a cell culture dish.

13. A method of differentiating stem cells into neural progenitor cells, the method comprising:
suspension-culturing the stem cells in a cell culture medium comprising a PKC inhibitor and a BMP inhibitor to obtain embryoid bodies (EBs); and
adherent-culturing cells obtained by chopping the EBs to induce differentiation into neural progenitor cells.

14. The method of claim 13, wherein the suspension culture is performed for 1 day to 10 days.

15. The method of claim 13, wherein the adherent culture is performed for 1 day to 15 days.
